(19) United States
(12) Patent Application Publication
(10) Pub. No.: US 2023/0023707 A1
(43) Pub. Date: Jan. 26, 2023

(54) REMOTE MONITORING OF RESPIRATORY FUNCTION USING A CLOUD-BASED MULTIMODAL DIALOGUE SYSTEM

(71) Applicant: Modality.AI, San Fransisico, CA (US)

(72) Inventors: Hardik Kothare, Burlingame, CA (US); Ramanarayanan Vikram, Berkeley, CA (US)

(73) Assignee: Modality.AI, San Fransisico, CA (US)

(21) Appl. No.: 17/552,351

(22) Filed: Dec. 15, 2021

Related U.S. Application Data
(60) Provisional application No. 63/223,424, filed on Jul. 19, 2021.

(51) Int. Cl. A61B 7/00; A61B 5/087; A61B 5/00; A61B 7/04; A61B 5/097; G16H 40/67

(52) U.S. Cl. CPC A61B 7/003; A61B 5/087; A61B 5/7278; A61B 7/04; A61B 5/097; A61B 5/4803; A61B 5/7465; A61B 5/0022; G16H 40/67

(57) ABSTRACT

A cloud or other network-based multimodal dialogue system is used to conduct automated screening interviews by engaging with conversational AI over a device of the user's choice (smartphone, tablet, laptop) from the comfort of their home. A screening interview will typically guide a user to blow towards a microphone, use signals from the microphone to calculate amplitudes, and use the amplitudes to calculate a flow rate and a flow volume. Contemplated systems and methods can be deployed in an automatically scalable cloud environment allowing it to serve an arbitrary number of end users at a very small cost per interaction. No special devices unique to the task are needed, which makes the technology accessible to a vast number of users. The technology can be natively equipped with real-time speech and video analytics modules that extract a variety of features of direct relevance to clinicians, thus allowing for measurement of multiple subsystems (motoric, phonatory, resonatory) in conjunction with lung function.

This application claims priority to U.S. provisional application Ser. No. 63/125,413 filed on 2020 Dec. 14. This and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is telemedicine.

### BACKGROUND

The following description includes information that may be useful in understanding the present invention. It is not an admission that any of the information provided herein is prior art or relevant to the presently claimed invention, or that any publication specifically or implicitly referenced is prior art.

Speech production depends on continuous expiration of air from the lungs. Each respiratory cycle during speech involves an exchange of larger volumes of air in the lungs as compared to quiet breathing. Weakness of respiratory muscles due to neurological conditions like Parkinson's Disease (PD) or Amyotrophic Lateral Sclerosis (ALS) may result in dysarthria; particularly, it may affect the overall loudness of speech. Lung function is thus key to efficient production of speech and is used as an objective measure for disease diagnosis and management by physicians and speech-language pathologists.

The clinical standard for measuring lung function is a spirometry test, and it involves the patient exhaling forcefully into a device which measures the flow of exhaled air. With telemedicine gaining traction in recent years, especially during the current COVID-19 pandemic, there is an increased need to make clinical tests available to patients at home. Remote spirometry would allow physicians to monitor lung function in patients longitudinally without having to schedule a visit to the clinic.

Previous work has demonstrated the feasibility of collecting spirometry data using signals generated from a microphone, without necessarily relying on auxiliary equipment. Examples of publications describing the prior art in this regard include US2015/0126888 to Patel and WO2016154139 to Patel.

One issue, however, is that considerable false readings in the resulting data can occur from user error. It is theoretically possible for a nurse or other professional to guide a user, but such a solution is impractical for obtaining readings from large numbers of users.

Thus, there is still a need for systems and methods that can guide collection of spirometry data using a microphone, without relying on humans guiding the users in collecting the data.

### SUMMARY OF THE INVENTION

The inventive subject matter provides apparatus, systems and methods in which a cloud or other network-based multimodal dialogue system is used to conduct automated screening interviews by engaging with conversational AI over a device of the user's choice (smartphone, tablet, laptop) from the comfort of their home. A screening interview will typically guide a user to blow towards a microphone, use signals from the microphone to calculate amplitudes, and use the amplitudes to calculate a flow rate and a flow volume.

Contemplated systems and methods can be deployed in an automatically scalable cloud environment allowing it to serve an arbitrary number of end users at a very small cost per interaction. No special devices unique to the task are needed, which makes the technology accessible to a vast number of users. The technology can be natively equipped with real-time speech and video analytics modules that extract a variety of features of direct relevance to clinicians, thus allowing for measurement of multiple subsystems (motoric, phonatory) in conjunction with lung function.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A is a schematic implementation of a method in which breathing data is calculated from a person breathing into a microphone.

FIG. 1B is an alternative portion of the schematic of FIG. 1 in which the person is breathing into a microphone other than a cell phone microphone.

FIG. 2 is a flow chart of steps in a preferred embodiment.

FIG. 3 is a Volume vs Time plot of breathing data obtained from a person blowing into a microphone.

### DETAILED DESCRIPTION

Throughout the following discussion, references will be made regarding a computing system. It should be appreciated that the use of this term is deemed to represent one or more computing devices having at least one processor configured to execute software instructions stored on a computer readable tangible, non-transitory medium. For example, a computing system can include one or more computers operating as a web server, database server, or other type of computer server in a manner to fulfill described roles, responsibilities, or functions.

The following discussion provides example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The recitation of ranges of values herein is merely intended to serve as a shorthand method of referring individually to each separate value falling within the range. Unless otherwise indicated herein, each individual value is incorporated into the specification as if it were individually recited herein, and ranges include their endpoints.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Unless a contrary meaning is explicitly stated, all ranges are inclusive of their endpoints, and open-ended ranges are to be interpreted as bounded on the open end by commercially feasible embodiments.

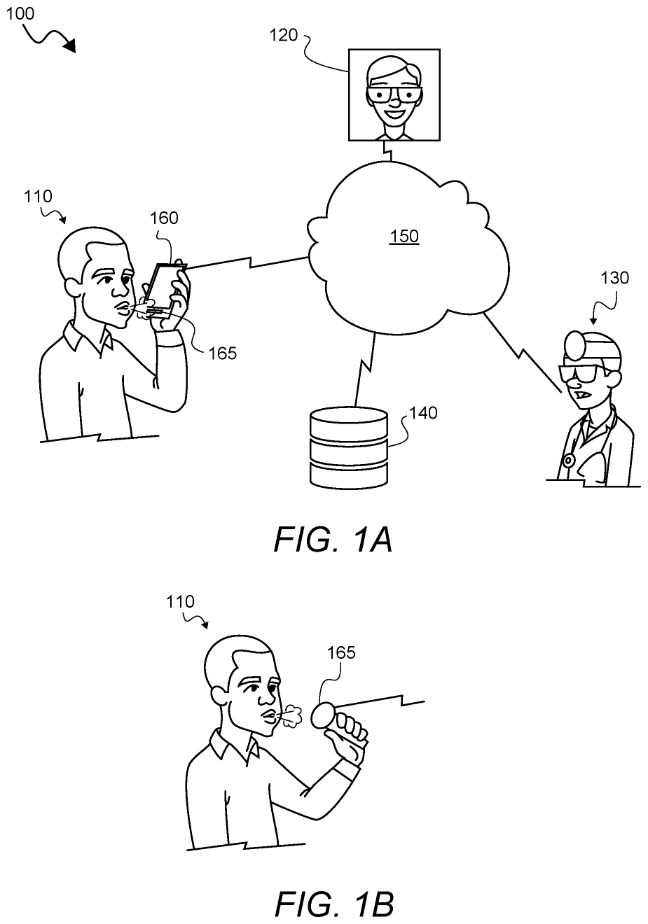

FIG. 1A is a schematic implementation of a method in which a virtual agent 120 guides a person 110 to blow into a microphone 160 to obtain breathing data. In FIG. 1A, the microphone 160 is incorporated into a cellphone. Signals from the microphone 160 are communicated through a network 150 to a computing system 140, which calculates at least one of breathing flow rate and flow volume from the microphone signals. Calculated flow rates and flow volumes calculated by the computing system 140 are sent to a medical professional or other service provider 130.

It is contemplated that the virtual agent can use any suitable manner to guide the person, including for example, using conversational or other audible speech, and written instructions.

FIG. 1B depicts an alternative portion of the schematic of FIG. 1, in which the person 110 is breathing into a microphone 165 other than the cell phone microphone 160. Microphone 165 should be interpreted as a generic microphone, including for example a hand-held stick microphone, a desktop microphone, and a webcam or other desktop or laptop microphone.

It is also contemplated that a camera could be used to estimate distances between the lips of the person and the microphone, while the person is blowing into the microphone. This can be done fairly easily when a camera is integrated into a device along with the microphone, as in a webcam. See e.g., https://photo.stackexchange.com/questions/40981/what-is-the-relationship-between-size-of-object-with-distance.

From a high level perspective, the method depicted by FIG. 1 comprises the following:
A user logs into a personalized and secure HIPAA compliant webpage (URL).
The user is then guided to set up his/her microphone, and greeted by a conversational AI (virtual) agent, who commences an audiovisual dialog interaction. Camera and audio calibration are also performed at this stage.
The virtual agent asks the user for non-identifiable details like age, sex, height and weight, along with other survey demographic information. This information can be used to perform vocal-tract modeling.
The agent guides the user through a structured interview, wherein he/she is instructed to perform specific tasks while audio and video is recorded (video in conjunction with computer vision can be used for user calibration which will reduce the error in estimation of lung function metrics).
The AI agent then instructs users to exhale forcefully into the microphone of their device and perform other guided spirometry tasks. The AI agent can nudge/guide the user and help with user cooperation. This is especially important because user error is the biggest problem in spirometry data collection.
Finally, the user is guided to the AI agent in filling out other questionnaires relevant to respiratory and general health.

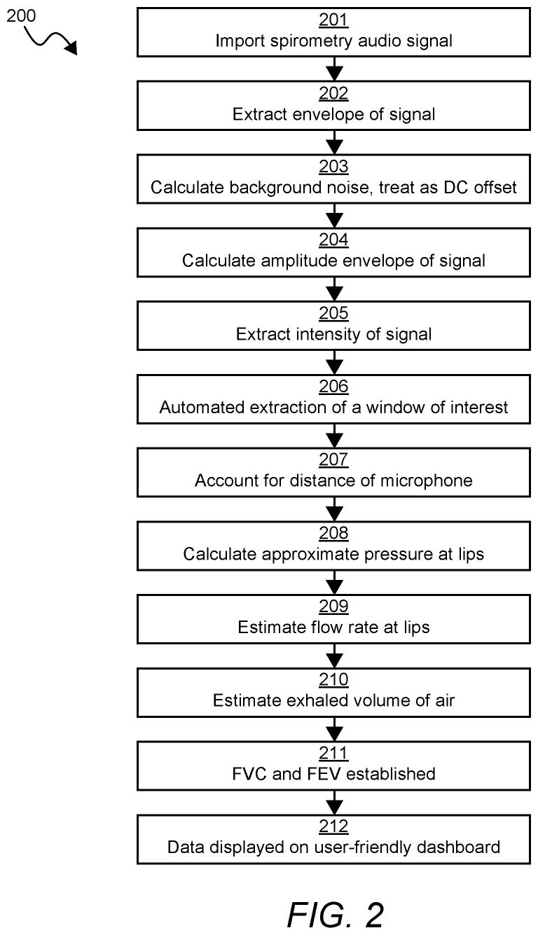

FIG. 2 depicts an exemplary method 200, comprising a series of steps 201-212 that derive breathing data from a person blowing into a microphone. Preferred embodiments of these steps 201-212 are detailed below.

Step 201—Import spirometry audio signal (.wav file) recorded by the platform as a Parselmouth (https://parselmouth.readthedocs.io/en/stable/index.html) object. Code can be as follows:

snd=parselmouth.Sound(‘test.wav’) # Replace ‘test’ by file name

Step 202—Extract the time domain envelope of the analytic signal using Hilbert transformation and add it back to the raw signal. Code can be as follows:

analytic_signal = scipy.signal.hilbert(2*abs(snd.values.T)).real
signal_plus_hilbert = (2*abs(snd.values.T)) + analytic_signal

Step 203—Calculate background noise and treat it as DC offset. Code can be as follows:

DCoffset=np.mean(signal_plus_hilbert[0:300])

Step 204—Smooth the resulting signal from step 202 using moving average and subtract DC offset from the smoothed signal to get the amplitude envelope of the signal (unit: Pascals). Code can be as follows:

```
def movingaverage (values,window):
  weights = np.repeat (1.0,window)/window
  smas = np.convolve(values,weights,‘valid’)
  return smas
averaged_signal_mic = np.zeros(len(signal_plus_hilbert))
averaged_signal_mic[1000:−1000] = movingaverage(signal_plus_hilbert
[:,0]-DCoffset,2001).real
```

Step 205—Extract intensity of the audio signal and repeat steps 203 and 204 for the intensity signal. Code can be as follows:

intensity = snd.to_intensity( )
intensity_DCoffset = np.mean(intensity.values.T[0:30])
averaged_intensity = np.zeros(len(intensity.values.T))
averaged_intensity[15:−15] = movingaverage(intensity.values.T
[:,0]-intensity_DCoffset,31).real

Step 206—Automated extraction of a window of interest (signal that is relevant) based on thresholding, i.e. find start time and end time and corresponding frames in the signal and use them as ‘frames of interest’. Code can be as follows:

start_time =
intensity.get_time_from_frame_number(np.where(averaged_intensity
[0:np.argmax(averaged_intensity)] <1)[−1][−1]+1)
end_time =
intensity.get_time_from_frame_number(np.where(averaged_intensity
[np.argmax(averaged_intensity):]<1)[0][0]−1+np.argmax
(averaged_intensity))
start_frame = int(snd.get_frame_number_from_time(start_time))
end_frame = int(snd.get_frame_number_from_time(end_time))
frames_of_interest = np.arange(start_frame,end_frame)

Step 207—Account for distance from the microphone by calculating a microphone distance factor which divides the maximum intensity by the 20*log of the maximum of the smoothed amplitude envelope in Pa. Microphone distance can be accounted for in any suitable manner, including via audio, or by measuring depth using computer vision. Code can be as follows:

microphone_distance_factor =
max(averaged_intensity)/(20*np.log10(max
(averaged_signal_mic)/0.00002))

Step 208—Calculate approximate pressure at lips by multiplying the smoothed amplitude envelope by the microphone distance factor. Code can be as follows:

averaged_signal=microphone_distance_factor*averaged_signal_mic

Step 209—Flow rate of air at lips or uₗᵢₚₛ(t) is estimated as done in previous studies, see e.g., Larson, E. C., et al. (2012, September). Proceedings of the 2012 ACM conference on ubiquitous computing (pp. 280-289).

uₗᵢₚₛ(t)˜2πrₗᵢₚₛ²\sqrt{2pₗᵢₚₛ₍ₜ₎}

where rₗᵢₚₛ is the radius of the lip aperture (as measured by a computer vision algorithm). Code can be as follows:

flow_at_lips = 2 * np.pi * np.square(lip_aperture) *
np.sqrt(averaged_signal[frames_of_interest])

Step 210—Exhaled volume of air is estimated by integrating the flow calculated in step 9 with respect to time. This gives us a volume by time curve. Code can be as follows:

volume = scipy.integrate.cumtrapz(averaged_signal[frames_of_interest],
x=snd.xs( )[frames_of_interest])

Step 211—The peak of the volume by time curve is Forced Vital Capacity (FVC). The volume of air exhaled in 1 second is Forced Expiratory Volume in 1 second (FEV₁). Code can be as follows:

forced_vital_capacity=max(volume)

FVC and FEV1 are displayed on a user-friendly dashboard accessible by clinicians and researchers (This dashboard also displays other multimodal metrics captured by Modality and is novel to our platform). Code can be as follows:

one_second_frame =
int(snd.get_frame_number_from_time(snd.get_time_from_frame_number
(frames_of_interest[np.where(volume > 0)][0])+1))
forced_expiratory_volume_one_second = volume[np.where
(frames_of_interest == one_second_frame)[0][0]]

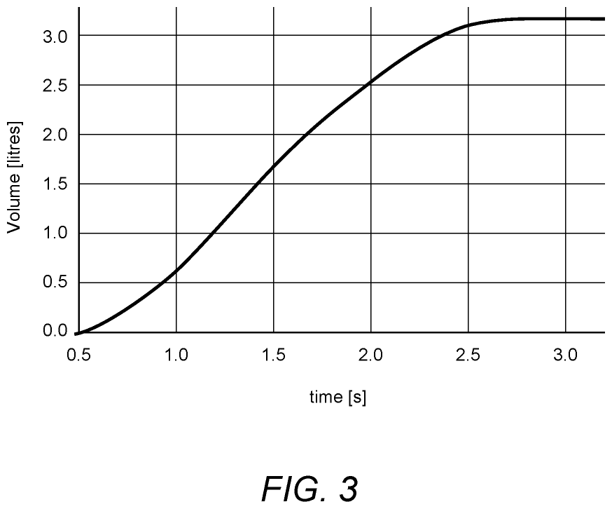

FIG. 3 is a Volume vs Time plot of breathing data obtained from a person blowing into a microphone, obtained where FVC=3.16 liters and FEV1=1.62 liters. The x axis starts where the audio signal is detected and ends where the audio signal ends. This graph provides real world evidence that breathing volume can be derived from microphone signals.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of deriving breathing information for a person, comprising:
using a virtual agent to guide the person to blow towards a microphone;
deriving a signal from the microphone;
using the signal to calculate an amplitude; and
using the amplitude to calculate a flow rate and a flow volume.

2. The method of claim 1, further comprising guiding the person to adjust a distance between the person's mouth and the microphone.

3. The method of claim 1, further comprising calculating approximate pressure at the lips of the person as a function of the amplitude and a microphone distance factor.

4. The method of claim 1, further comprising guiding the person to blow towards the microphone through a tube.

5. The method of claim 1, further comprising using the flow rate to calculate the flow volume.

6. The method of claim 1, wherein the amplitude comprises an amplitude envelope.

7. The method of claim 6, using the amplitude envelop to calculate the flow rate and the flow volume.

8. The method of claim 1, further comprising determining a Forced Vital Capacity (FVC) as a peak in the flow rate over time.

9. The method of claim 1, further comprising determining a Forced Expiratory Volume (FEV) as a peak in the flow volume over period of time.

10. The method of claim 1, further comprising the virtual agent using conversational speech to guide the person.

11. The method of claim 1, further comprising the virtual agent using audible speech to guide the person.

12. The method of claim 1, further comprising the virtual agent using written instructions to guide the person.
